(19) United States
(12) Patent Application Publication
(10) Pub. No.: US 2022/0162538 A1
(43) Pub. Date: May 26, 2022

(54) CONTAINER FOR STORING, MIXING AND/OR CULTIVATING A MEDIUM

(71) Applicant: Sartorius Stedim Biotech GmbH, Göttingen (DE)

(72) Inventors: Christian Grimm, Heilbad Heiligenstadt (DE); Andreas Prediger, Göttingen (DE); Matthias Bode, Göttingen (DE); Arkadiusz Pajak, Wiltshire (GB)

(73) Assignee: Sartorius Stedim Biotech GmbH, Göttingen (DE)

(21) Appl. No.: 17/441,136

(22) PCT Filed: Mar. 19, 2020
(86) PCT No.: PCT/EP2020/057633

§ 371 Date: Sep. 20, 2021

(30) Foreign Application Priority Data
Mar. 21, 2019 (DE) 10 2019 001 995.4

(51) Int. Cl. C12M 1/06; C12M 1/00; C12M 1/34; C12M 1/12

(52) U.S. Cl. CPC C12M 27/02; C12M 23/28; C12M 41/12; C12M 37/00; C12M 41/30; C12M 41/40; C12M 41/46; C12M 41/26

(57) ABSTRACT

A container for storing, mixing and/or cultivating a medium, in particular a bioreactor for a medium, comprises a line system as a discharge line, feed line and/or bypass line of the bioreactor. The system can comprise a line body formed as a single piece for a medium to flow therethrough. The has a first and a second connection region for connecting in particular to the container and/or the line system and at least a first and a second coupling apparatus in the region between the connection regions.

The invention relates to a container for storing, mixing and/or cultivating a medium, in particular a bioreactor for a medium, comprising a line system, a line body of such a line system, and a measuring system for a container for storing, mixing and/or cultivating a medium, in particular a bioreactor.

Bioreactors are used, for example, in breweries, wineries and the pharmaceutical and cosmetic industries for cultivating media. Such media can be provided in single-use bags or in reusable vessels which can have a volume of several hundred liters. The biological media are introduced into a container (e.g., bioreactor) and maintained at a predeterminable temperature over a predetermined period of time of usually several hours and cultivated optionally with the addition of oxygen. It is vitally important to reliably monitor the cultivating process, especially by carrying out different measurements on the biological medium. Since a bioreactor is usually handled in a sterile environment, particularly high requirements are placed on the performance of such measurements or on the measuring systems used in order to ensure the quality control of the medium. It is also advantageous if the elements of the measuring system coming into contact with the media can be sterilized.

Frequently, measurements of the medium are carried out by different measuring components in a line system which discharges the medium to be cultivated from the container, such as a bioreactor. For this purpose, the various reusable measurement components are generally connected to a “single-use” line system. Measurement systems for such single-use line systems therefore generally comprise a plurality of individual measurement components for measuring certain parameters of a medium. This plurality of measurement components is integrated into the single-use line system by connecting the individual sensor elements to separate or external line components of the line system. However, this procedure involves an increased risk of leakages, in particular by incorrect installation of the individual connection points. The connection and the prior sterilization of the individual measuring components are also labor-intensive and cost-intensive. In addition, a measuring component already connected to the line system can be exchanged, for example in the event of a defect, only with great effort.

It is, therefore, an object of the present invention to provide a container for storing, mixing and/or cultivating a medium, in particular a bioreactor for a medium, comprising a line system and a measuring system for a container for storing, mixing and/or cultivating a medium, in particular a bioreactor, which allows high variability in measurements and at the same time increases process reliability.

The aforementioned object is achieved by the subject matter of the independent claims. Advantageous embodiments form the subject matter of the dependent claims.

In particular, the present invention provides a line body for a line system of a container for storing, mixing and/or cultivating a medium, in particular a bioreactor, which is easy to handle in order to simplify proper assembly and reduce the risk of leakages. In addition, a measuring system equipped with the line body advantageously enables a simple mounting of different sensors so that a reduction in the wide variety of single-use components of the measuring system that are to be produced is achieved. In addition, the line body enables a simplification of the production method and of the sterilization process.

The invention relates to a container for storing, mixing and/or cultivating a medium, in particular a bioreactor for a medium, comprising a line system as a discharge line, feed line and/or bypass line of the container, the line system at least comprising a line body, which is formed as a single piece and is suitable for a medium to flow therethrough, the line body having a first and a second connection region for connecting in particular to the container and/or the line system and at least a first and a second coupling apparatus in the region between the connection regions, the first and second coupling apparatuses being structurally different and being designed to be coupled to respective structurally different measuring apparatuses.

A container within the meaning of this invention is a vessel, container or boiler which is particularly suitable for storing, mixing, controlling the temperature of and/or cultivating a medium. In particular, such a container may be suitable for use as a single-use or reusable bioreactor.

The line system of the container is in particular suitable for draining or discharging a medium from the container and/or for feeding a medium into the container or guiding it into the container. Alternatively and/or additionally, the line system may have a loop line/bypass line which diverts a medium located in the bioreactor, i.e., guides it out of the container and returns it to the container at another location. Such a bypass line is advantageous in particular because a medium of the container can be guided through a measuring system located outside the container in order to monitor the cultivation process of the medium and optionally to adjust the process parameters. The medium of the container is, for example, a liquid, a solution, a suspension, a dispersion, an emulsion, a heterogeneous/homogeneous mixtures of substances and/or a gas. The preferably substantially rectilinear and/or tubular/round line body has at both ends a connection region on, at or with which the line body can be connected to further components of the line system or attached to them in a substantially leak-tight manner. Alternatively and/or additionally, the line body can be connected to the container or be directly attached thereto. In particular, the line body can be provided in the line system or connected to the line system/the container without coupled measuring apparatuses.

The line body preferably has at least a third coupling apparatus. The first, second and third coupling apparatuses are particularly preferably structurally different. Particularly preferably, each of the coupling apparatuses of the line body is designed in such a way that only one specific measuring apparatus can be coupled to them in each case. Particularly preferably, the line body furthermore has a fourth coupling apparatus. More preferably, the first, second, third and fourth coupling apparatuses are structurally different. Two or three of the four coupling apparatuses may also be of similar design. This enables, for example, two or three identical measuring apparatuses to be mounted on the line body in order to enable redundancy measurements. Such a redundant measurement preferably takes place in a front and a rear region of the line body in order to obtain increased measurement accuracy. More preferably, the line body can have five, six, seven or more coupling apparatuses, wherein redundant coupling apparatuses can be present.

The coupling apparatuses are advantageously arranged at a distance from one another in the circumferential direction of the line body and/or in the flow direction of a medium flowing through the line body. Particularly preferably, the coupling apparatuses are equally spaced apart and arranged next to one another in the flow direction of a medium flowing through the line body.

The measuring apparatuses that can be coupled to the coupling apparatuses of the line body are preferably particularly suitable for measuring the temperature, pressure, flow rate, oxygen content, pH value, conductivity, viscosity and/or optical parameters of a medium flowing through the line body. Additionally and/or alternatively, measuring apparatuses or sensors other than the aforementioned measuring apparatuses or sensors can also be coupled to the coupling apparatuses in order to detect further parameters of the medium. In this case, the different measuring apparatuses preferably have different structural features so that a measuring apparatus can only be coupled to a coupling apparatus intended for said measuring apparatus. Particularly preferably, the coupling apparatuses have different optical markings or features so that an assignment of the measuring apparatus to an associated coupling apparatus can be facilitated and thus the ease of handling can be further simplified and process reliability can be further increased. A color code and/or structural “key-lock” elements are particularly suitable for this purpose.

The container, the line system and/or the line body is or are preferably intended for one-time use (so-called “single-use” component). As described, single-use components are used where there are increased requirements relating to sterility so that contamination of the cultivated medium can be substantially prevented or reduced. Such a single-use container, in particular such a single-use bioreactor, consists, for example, of composite film comprising polyester (PE), polyamide (PA), polypropylene (PP), ethylene-vinyl acetate (EVA), ethylene-vinyl alcohol copolymer (EVOH), and/or polyvinyl chloride (PVC). The line system and/or the line body preferably consist substantially of thermoplastic materials, such as polyethylene (PE), polypropylene (PP), polyethylene terephthalate (PET), polybutylene terephthalate (PBT), fluoropolymers and similar polyolefins.

The invention further relates to a line body for a line system for discharging, supplying and/or diverting a medium of a container, the line body being formed as a single piece and having a first and a second connection region for connecting the line body, in particular to a container and/or to the line system, and at least a first and a second coupling apparatus in the region between the connection regions, the first and second coupling apparatuses being structurally different and being designed to be coupled to respective structurally different measuring apparatuses. The line body may in particular be part of a line system of a container such as described above and in particular have the features and properties described above.

The line body formed as a single piece is preferably substantially rectilinear and/or tubular/round. In particular, the line body advantageously has substantially no influences impeding flow, such as cross-sectional changes, baffles, deflections, undercuts and small dead volumes. The flow body preferably comprises a wall comprising thermoplastic materials, such as polyethylene (PE), polypropylene (PP), polyethylene terephthalate (PET), polybutylene terephthalate (PBT) and similar polyolefins. The line body is preferably suitable for being produced from correspondingly suitable materials by injection molding. The flow body is more preferably suitable for being produced at least partially using an additive manufacturing method, such as stereolithography (SL), laser sintering (LS/SLS) and/or fused deposition modeling (FDM), from materials suitable for this purpose, such as plastics (thermoplastics, such as polyethylene, polypropylene, polylactide, ABS, PETG and thermoplastic elastomers), synthetic resins, ceramics and metals. More preferably, the line body consists of inert materials and/or has a coating of the inner wall made of inert materials so that any influence by the medium flowing through the line body is substantially prevented.

The line body preferably has at least a third coupling apparatus. More preferably, the first, second and third coupling apparatuses of the line body are structurally different. More preferably, the line body has four or more coupling apparatuses.

The measuring apparatuses that can be coupled to the coupling apparatuses of the line body are advantageously particularly suitable for measuring the temperature, pressure, flow rate, oxygen content, pH value, conductivity, viscosity and/or optical parameters of a medium flowing through the line body.

The line body is more preferably intended for one-time use or is a single-use line body. The line body preferably consists of material comprising thermoplastic materials, such as polyethylene (PE), polypropylene (PP), polyethylene terephthalate (PET), polybutylene terephthalate (PBT) and similar polyolefins.

According to a particularly preferred embodiment, the line body has coupling apparatuses of different designs. In particular, at least one region of a coupling apparatus has a wall thickness of less than approximately 3 mm, preferably less than approximately 2 mm. Such an embodiment of the coupling apparatus enables, for example, a reduction in measurement noise or inaccuracies caused by the wall of the line body. More preferably, at least one region of a coupling apparatus alternatively and/or additionally has a thermal conductivity in order to enable measurements of the temperature of the medium from outside the line body. Alternatively and/or additionally, at least one region of a coupling apparatus has a coating on the outside of the line body, particularly preferably comprising TPE (thermoplastic elastomers), silicone, NBR (acrylonitrile butadiene rubber), or materials having similar properties. Such a coating in particular enables an advantageous mechanical coupling of the measuring apparatuses to the coupling apparatuses embodied in this way. Alternatively and/or additionally, a region of a coupling apparatus can have an optical transmittance in order to enable measurements of optical parameters of the medium from outside the line body. Such a region is comprises preferably permeable to electromagnetic radiation, in particular to radiation with a wavelength in the range between approximately 120 nm and approximately 50 μm. In particular, this allows measurements relating to UV absorption, turbidity, scattering and other spectroscopic measurements of the medium. More preferably, at least one region of a coupling apparatus may alternatively and/or additionally be permeable to sound in order to enable measurements of the flow rate of the medium from outside the line body. Such a region preferably comprises a material with a high transmittance so that sound waves can pass through the coupling apparatus, with little or substantially no loss of intensity, and reach the medium inside the line body. More preferably, a region of a coupling apparatus partially comprises materials having high reflectivity (acoustic and/or optical) in order to achieve an increase in the measurement distance. In particular, a reflective surface can be mounted in a region of the line body which is opposite a region in which optical or acoustic radiation is introduced into the line body.

A coupling apparatus of the line body preferably has one or more openings in the line body for receiving at least a portion of a measuring apparatus or for inserting at least a portion of a measuring apparatus into the interior of the line body. Such an opening is especially suitable for receiving or containing or inserting measuring apparatuses which required direct contact with the medium or for which such a contact is advantageous, such as temperature sensors, flow sensors and conductivity sensors. Alternatively, such an opening can be used to implement an outlet for sampling, a valve and/or a metering device.

A coupling apparatus of the line body preferably has a deflectable membrane in order to enable measurements of the pressure inside the line body or the medium from outside the line body, wherein the deflectable membrane preferably has: a first side which faces the interior of the line body and can come into contact with the medium inside the line body; and a second side which is accessible from outside the line body. Such a membrane is preferably mounted in a substantially leak-tight manner in or on a recess or opening of the wall of the line body. As an alternative to a membrane, other devices which are suitable for making the pressure prevailing inside the line body measurable from outside the line body, such as a spring-loaded pin, can also be considered.

More preferably, a coupling apparatus of the line body has two or more electrodes, wherein a first side of the two or more electrodes faces the interior of the line body and can come into contact with the medium inside the line body, and a second side of the two or more electrodes is accessible from outside the line body. The electrodes are particularly preferably arranged at a distance from one another in the direction of flow of the medium. A measuring apparatus coupled to the two or more electrodes is especially suitable for measuring the electrical conductivity of the medium.

A coupling apparatus of the line body advantageously has a receiving apparatus, in particular a shaft and/or a rail, for receiving at least a portion of a measuring apparatus. Such a receiving apparatus is preferably formed integrally with the wall of the line body and enables a measuring apparatus to be easily and properly positioned and/or mounted on the coupling apparatus.

The line body is more preferably suitable for irradiation sterilization and/or autoclave sterilization or steam sterilization. This enables a particularly user-friendly integration of the line body in a line system or connection of the line body to a container, since the line body can be provided already presterilized. Moreover, the measuring apparatuses can only be coupled to the coupling devices once the line body or the line system has been installed so that the sterility inside the line body or the line system is not affected. In particular, this allows multiple measuring apparatuses to be used and thus allows significant cost and resource savings.

The line body advantageously has coupling apparatuses which are arranged at a distance from one another in the direction of flow of a medium flowing through the line body and/or in the circumferential direction of the line body. Two coupling apparatuses arranged substantially radially opposite one another are particularly preferred.

More preferably, the line body further has, in the medium flow region of the line body, a geometry which is suitable for influencing the flow properties of the medium. Particularly preferably, the region has hydrodynamic and/or aerodynamic structures which create a flow type which substantially expands in the line direction.

Advantageous is a line body which comprises a line main body with the connection regions and with a cutout, and a line sub-body having at least two coupling apparatuses, wherein the cutout of the line main body is embodied in such a way or designed to receive the line cable body at least in regions. A substantially leak-tight joining of the line main body and the line sub-body is advantageous in this case. Alternatively, the line main body and line sub-body may each have at least one coupling apparatus.

The invention further relates to a measuring system for a container for storing, mixing and/or cultivating a medium, in particular a bioreactor, for measuring parameters of a medium, comprising a line body as described above and two or more measuring apparatuses, each of which is coupled, preferably detachably, to one of the coupling apparatuses of the line body, and wherein at least two of the measuring apparatuses are structurally different. The measuring system is preferably part of a line system for a container for storing, mixing and/or cultivating a medium, in particular a bioreactor, preferably comprising a line body formed as a single piece, as described above. Particularly preferably, the line body of the measuring system has four coupling apparatuses, wherein at least three of the coupling apparatuses are structurally different.

Particularly preferably, the measuring system comprises four different coupling apparatuses and four measuring apparatuses coupled thereto. The respective coupling apparatuses and measuring apparatuses coupled thereto are advantageously suitable for detecting the following parameters of the medium flowing through the line: pressure, flow rate, conductivity and temperature. Additionally and/or alternatively thereto, the measuring system has coupling apparatuses and measuring apparatuses that are suitable for measuring the viscosity and/or optical parameters, such as UV absorption, turbidity, scattering, and spectroscopic parameters. Exemplary embodiments of particularly suitable developments of the coupling apparatuses of the line body are described above.

In the following, individual embodiments for achieving the object are described by way of example with reference to the figures. Some of the individual described embodiments have features which are not absolutely necessary for carrying out the claimed subject matter but which, in certain applications, provide desired properties. Thus, embodiments which do not have all the features of the embodiments described below should also be considered disclosed as coming under the technical teaching described. Furthermore, in order to avoid unnecessary repetitions, certain features are mentioned only with respect to one of the embodiments described below. It should be noted that the individual embodiments are therefore to be considered not only in their own right, but also in a combination. On the basis of said combination, the person skilled in the art will recognize that individual embodiments may also be modified by including individual or multiple features of other embodiments. It should be noted that a systematic combination of the individual embodiments with individual or multiple features that are described with respect to other embodiments, may be desirable and expedient, and is therefore to be considered and also regarded as encompassed by the description.

### BRIEF DESCRIPTION OF THE DRAWINGS

FIG. 1 shows an exemplary embodiment of a container for storing, mixing and/or cultivating a medium, for example a bioreactor, comprising a line system as a discharge line, feed line and bypass line, wherein the line system comprises a line body formed as a single piece;

FIG. 2 shows a perspective view of an exemplary embodiment of a line body for a line system having four coupling apparatuses and a measuring apparatus coupled to one of the coupling apparatuses;

FIG. 3 shows a perspective view of an exemplary embodiment of a measuring system for a container for storing, mixing and/or cultivating a medium, having a line body comprising four coupling apparatuses and four measuring apparatuses coupled thereto;

FIG. 4 shows a section of an exemplary embodiment of a line body having a coupling apparatus and a coupled measuring apparatus for measuring the temperature of a medium;

FIG. 5 shows a section of a further exemplary embodiment of a line body having a coupling apparatus and a coupled measuring apparatus for measuring the conductivity of a medium.

FIG. 6 shows a further exemplary embodiment of a line body of a measuring system having two coupling apparatuses arranged opposite one another;

FIG. 7 shows an alternative exemplary embodiment of a line body, wherein two coupling apparatuses are arranged one behind the other at a distance from one another in the direction of flow of the medium;

FIG. 8 shows an exemplary embodiment of a line body having a line main body and a line sub-body having two coupling apparatus;

FIG. 9 shows a longitudinal section of a line body according to the embodiment in FIG. 8, wherein the line body has a flow-shaping geometry in the medium flow region;

FIG. 10 shows a further exemplary embodiment of a line body having a coupling apparatus comprising a coating;

FIG. 11A shows a longitudinal section of a line body according to one embodiment of FIG. 10;

FIG. 11B shows an alternative embodiment of the line body of FIG. 10 in longitudinal section;

FIG. 11C shows a further alternative embodiment of the line body of FIG. 10 in longitudinal section.

### DETAILED DESCRIPTION OF THE DRAWINGS

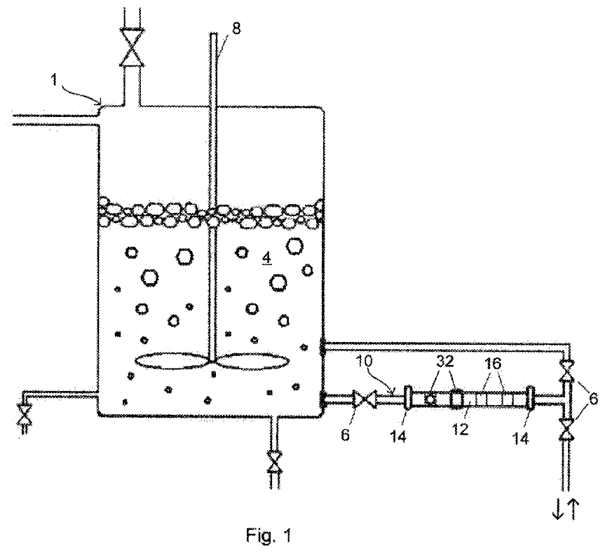

FIG. 1 shows a container 1 for storing, mixing and/or cultivating a medium, for example a bioreactor, according to a preferred embodiment of the invention. The container 1 shown is a single-use container consisting, for example, of polyester (PE), polyamide (PA), polypropylene (PP), ethylene-vinyl acetate (EVA), ethylene-vinyl alcohol copolymer (EVOH), and/or polyvinyl chloride (PVC). Alternatively, a reusable reactor made of glass and/or metal/stainless steel, for example, is also suitable (e.g., UniVessel® Glass/SU). Alternatively, such a container can be provided in a platform (e.g., Ambr®250) or in a housing (e.g., FlexSafe STR® with BIOSTAT STR® system) or used together with a monitoring unit (e.g., UniVessel® Glass/SU with BIOSTAT® A/B/Cplus, FlexSafe® RM with BIOSTAT® RM). The container 1 contains a medium 4, wherein the medium 4 is preferably a liquid, a solution, a suspension, a dispersion, an emulsion and/or a heterogeneous/homogeneous mixture of substances. The container 1 preferably has a stirring device 8 for circulating the medium 4 and one or more feed and/or discharge lines for feeding or discharging media. The container 1 in accordance with the preferred embodiment shown further comprises a line system 10. The line system 10 can in particular comprise one or more valves 6 and/or be connected to the container 1 by means of such valves. The line system 10 can be designed as a feed line, a discharge line and/or a bypass line. In accordance with the preferred embodiment shown, the line system 10 comprises a plurality of valves for controlling a flow of a medium in the line system 10 so that a medium can be selectively fed from the line system 10 to the container 1 or discharged from the container 1. Diverting of the medium 4 of the container 1 is also possible, wherein the medium 4 is guided out of the container 1 at one location thereof and is guided back in at another location of the container. Further possible embodiments of a feed/discharge/bypass line comprise filtration lines, for example having filter modules (alternating tangential flow filtration (ATF)/tangential flow filtration (TFF)), in order to discharge certain filtered constituents of the medium and to feed other constituents of the medium back to the container. The line system 10 preferably consists substantially of thermoplastic materials, such as polyethylene (PE), polypropylene (PP), polyethylene terephthalate (PET), polybutylene terephthalate (PBT) and similar polyolefins. The line system 10 may in this case comprise different components, in particular line components, valves and/or connection or attachment components.

The line system 10 shown in FIG. 1 further comprises a line body 12 formed as a single piece through which medium can flow, which flows or streams from and/or to the container 1. The line body 12 is preferably substantially tubular and/or rectilinear. However, it may also be curved and/or oval, for example. The line body 12 has connection regions 14 at both ends via or with which the line body 12 can be connected to other components of the line system 10 or attached thereto. Sterile connectors (e.g., Lynx® connector, Opta® connector) are preferably used to integrate the line body 12 into the line system 10 and/or to achieve a substantially leak-tight and sterile connection between the components of the line system 10 and the line body 12. Alternatively and/or additionally, the line body 12 may, for example, be connected to the container 1 and/or to an external line. The line body 12 may also alternatively and/or additionally be connected, for example, to a connector clamp (Tri-Clamp), an adapter, a coupling (BSP), a hose barb, a quick connector, a plug-in nipple and/or a thread. Suitable seals or sealants can preferably be used to obtain a substantially leak-tight connection between the individual components of the line system 10 and/or to the container 1. One or more connection regions 14 of the line body 12 may be designed to be substantially identical or different. In particular, the type, diameter, length and/or shape may vary as desired. Likewise, the diameter of one or more parts of the connection regions 14 may differ to a greater or lesser degree from a diameter of the line body 12.

The preferred embodiment of the line body 12 shown in FIG. 1 has a plurality of, for example, four coupling apparatuses 16, wherein a measuring apparatus 32 is coupled respectively to two of the four coupling apparatuses 16. The number and/or type of measuring apparatuses 32 coupled to the line body 12 may vary depending on the application. The coupling apparatuses 16 of the line body 12 are arranged at a distance from one another (preferably at substantially regular distances) in the longitudinal direction of the line body 12 or along a direction of flow of a medium flowing through the line body 12. The coupling apparatuses 16 are preferably structurally different from one another to allow an advantageous association between a coupling apparatus 16 and a correspondingly designed measuring apparatus 32. It is thus advantageously possible to mount or couple different measuring apparatuses 32 on a respective corresponding coupling apparatus 16.

A coupling apparatus 16 is particularly preferably designed in such a way that it is suitable for advantageous coupling with different measuring apparatuses 32. The measuring apparatuses 32 coupled to or mounted on the coupling apparatuses 16 of the line body 12 are especially suitable for measuring the temperature, pressure, flow rate, conductivity, viscosity and/or optical parameters of a medium flowing through the line body 12. The line body 12 thus represents a universal component of the line system 10 for mounting a plurality of measuring apparatuses 32 in a plurality of possible combinations. Some exemplary and particularly preferred developments of coupling apparatuses 16 and measuring apparatuses 32 are described below.

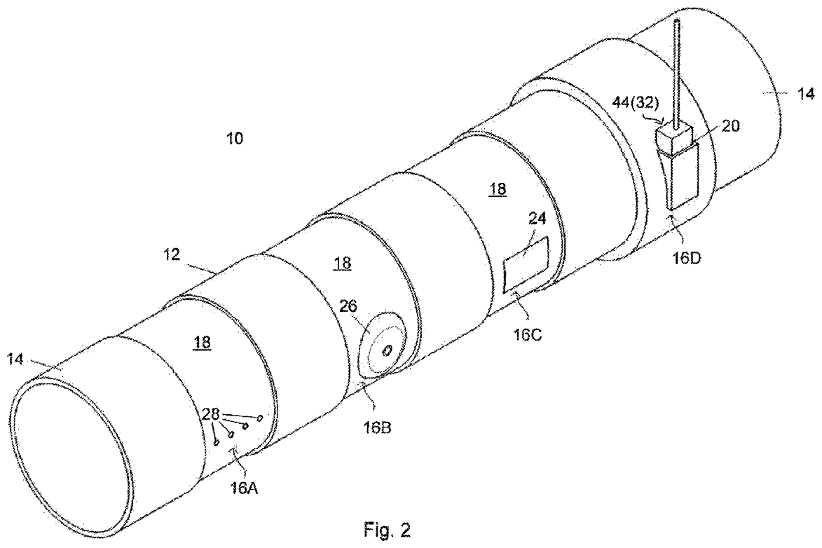

FIG. 2 shows an exemplary and preferred embodiment of a line body 12 of a line system 10, which line body is formed as a single piece and is substantially tubular. The line body 12 is part of a line system 10 or is provided as part of a line system 10 which can feed a medium to a container 1 and/or discharge it from a container 1. The line body 12 is preferably designed to be sterilizable (for example, by gamma radiation and/or autoclaving). The line body 12 has at both ends a connection region 14 through which the line body 12 can be connected to components of the line system 10 and/or to a container or can be integrated into the line system. The line body 12 has at least two (e.g., four) coupling apparatuses 16A-16D, wherein the coupling apparatuses 16A-16D are preferably structurally different. In the embodiment shown, the coupling apparatuses 16 are arranged along the longitudinal axis of the line body 12 at substantially equal distances from one another.

In the preferred embodiment shown, the coupling apparatus 16A has a plurality of (for example, four) electrodes 28 which are introduced into the wall of the line body 12. The individual electrodes 28 preferably extend from the inner circumference of the line body 12 to its outer circumference, so that the electrodes 28 correspond to a through-connection through the line body 12. The electrodes 28 are accessible from outside the line body 12 so that a measuring apparatus 32 having correspondingly designed contact elements can tap signals from the electrodes 28 or transmit signals to the electrodes 28. The electrodes 28 are preferably embedded in the wall of the line body during its production. Alternatively, electrodes can be introduced into openings in the wall of the line body. The electrodes can terminate flush with the inner and/or outer circumference of the line body or protrude from the wall of the line body. The electrodes preferably comprise rust-free steel/stainless steel, platinum and/or titanium or materials having similar properties, in particular similar electrical conductivity. The materials are preferably substantially (chemically) inert with respect to the constituents of the medium or have substantially no cell damaging influence on constituents of the medium. The coupling apparatus 16A is particularly suitable for being coupled to a conductivity sensor in order to measure the conductivity of a medium located in the line body 12 or flowing through the line body 12. Moreover, the coupling apparatus 16A preferably has, at least in regions, a surface coating 18 which provides a soft contact surface for an advantageous mechanical coupling of a measuring apparatus 32, in particular of fastening devices for mounting on the coupling apparatus 16 of the line body 12. Furthermore, in the preferred embodiment shown, the coupling apparatus 16 has a thinner wall compared to the line body 12. The wall of the coupling apparatus 16 is preferably smaller than approximately 2 mm. As a result, the coupling apparatus 16 is further structurally distinct or different from the line body 12.

A further particularly preferred embodiment of a coupling apparatus 16 shows the coupling apparatus 16B which has a deflectable membrane 26 inserted into the line body 12. The membrane 26 preferably comprises TPE (thermoplastic elastomers), silicone, NBR (acrylonitrile-butadiene rubber), or materials having similar properties. The diaphragm 26 is in contact with the medium flowing through the line body 12 and is deflected or deformed according to the pressure located in the line body 12 (preferably substantially in the radial direction of the line body 12). The coupling apparatus 16B is in particular suitable or designed to be coupled to a pressure sensor that can measure or determine the pressure transmitted by the membrane 26 and/or the deflection of the membrane 26.

The diaphragm 26 is preferably inserted at least partially into a recess or opening of the line body 12 and sealed toward the line body 12. The membrane 26 is preferably designed in such a way that the maximum deflection of the membrane 26 is limited in order to prevent a defect in the membrane 26, e.g., if no measuring apparatus 32 is coupled to the membrane 26 during operation of the line system 10. The coupling apparatus 16B further preferably has a surface coating 18 in order to enable an advantageous mechanical coupling to a measuring apparatus to be coupled. A measuring apparatus coupled to the membrane 26 can preferably measure or detect the deflection of the membrane 26 or the pressure transmitted from the membrane 26 to the measuring apparatus 32. Piezoelectric, capacitive, inductive and/or piezoresistive sensors are especially suitable as the measuring apparatus 32. As an alternative to the illustrated membrane 26, other devices which are suitable for making the pressure prevailing inside the line body 12 measurable or detectable from outside the line body 12, such as a spring-loaded pin, which protrudes substantially radially from the line body 12, also come into consideration.

The coupling apparatus 16C shows a further exemplary and particularly preferred embodiment of a coupling apparatus 16. The coupling apparatus 16C has at least one optical window 24, for example in the form of a glass substrate or a glass pane. The optical window 24 can, for example, be introduced or inserted into a cutout or recess in the line body 12 in a substantially leak-tight manner. The optical window 24 shown enables, for example, the introduction of electromagnetic radiation into the interior of the line body 12 and/or the detection of electromagnetic radiation emitted from the interior of the line body 12 through the optical window. Particularly preferred materials for such an optical window 24 are quartz, sapphire, borosilicate. In addition, the optical window 24 may comprise one or more different coatings, in particular optical filters.

The optical window 24 according to a preferred embodiment has an optical transmittance that allows optical measurement of the medium located within the line body 12. Electromagnetic radiation of a specific (predetermined or predeterminable) frequency can preferably be conducted via the optical window 24 (preferably substantially without intensity losses) into the interior of the line body 12 in order to interact with the medium. Radiation emerging from the inside of the line body 12 can also be detected outside the line 1.

In accordance with the preferred embodiment shown, the optical window 24 is substantially transparent to radiation having a wavelength in the range between approximately 120 nm and approximately 50 μm.

The coupling apparatus 16C is particularly suitable for being coupled to an optical measuring apparatus 32 in order to carry out measurements of optical parameters of the medium within the line body 12. Such an optical measuring apparatus 32 preferably has a device for introducing electromagnetic radiation and/or at least one optical sensor for detecting electromagnetic radiation.

As an alternative to the embodiment shown, a coupling apparatus 16, suitable in particular for optical measurements, may have a second optical window 24 which is provided substantially radially opposite the first optical window 24 in the line body 12, so that electromagnetic radiation can be introduced by a measuring apparatus 32 through one of the optical windows 24 and emerge through the (preferably substantially opposite) further optical window 24 in order to be detected by a measuring apparatus 32 coupled or arranged on said window 24. Such an advantageous embodiment thus enables the medium to be “illuminated” inside the line body 12 in order to determine, for example, a degree of absorption of light (e.g., at least of a specific wavelength) by the medium in the line body 12. Alternatively or additionally, the introduction and detection can take place by the same measuring apparatus 32.

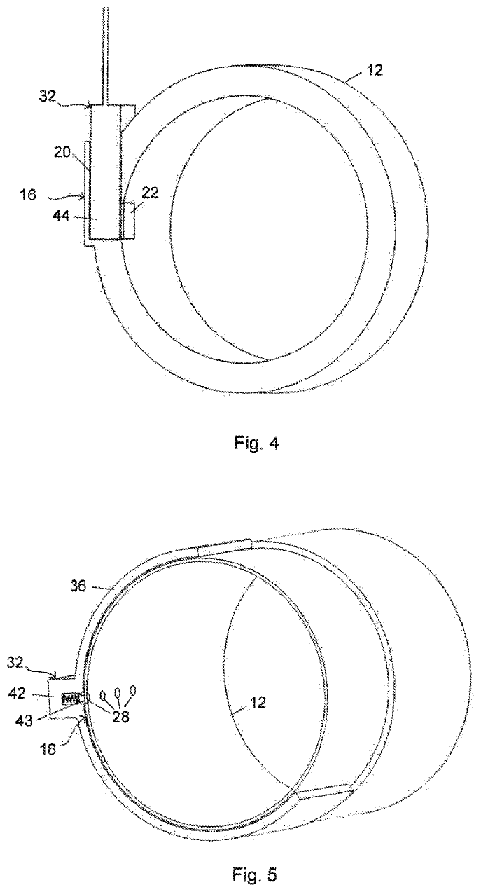

A further preferred embodiment of a coupling apparatus 16 shows the coupling apparatus 16D which has a holding or receiving apparatus 20 in the form of a shaft or a pocket. Alternatively, the receiving apparatus 20 may have differing features, in particular those which enable or simplify the mounting and/or positioning of a measuring apparatus 32 on the coupling apparatus 16 of the line body 12, such as a rail and/or a clip. The holding or receiving apparatus 20 shown is, in particular, suitable for at least partially receiving or holding a measuring apparatus 32. The measuring apparatus 32 can be detachably or non-detachably mounted or held in the receiving apparatus 20. The receiving apparatus 20 of the coupling apparatus 16D is preferably designed in such a way that a measuring apparatus 32 positioned in or on the receiving apparatus 20 is coupled to a region of the coupling apparatus 16D that is advantageous for the respective measurement. For example, a temperature sensor positioned in or on the receiving apparatus 20 is coupled to a region of the coupling apparatus 16D which allows a particularly precise measurement of the temperature of the medium due to its thermal conductivity. A corresponding particularly preferred embodiment is shown in FIG. 4. The coupling apparatus 16C of the preferred embodiment shown furthermore has a wall which has a greater thickness in comparison to the wall of the line body 12 and as a result is further structurally different from the line body 12.

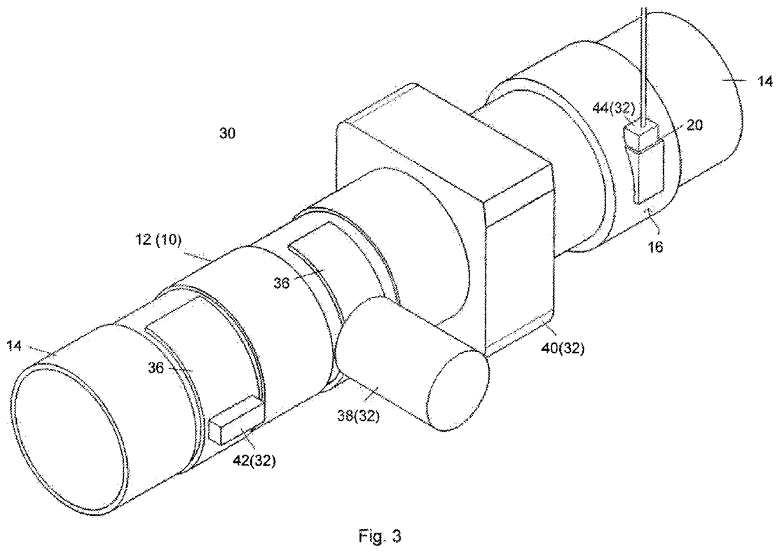

FIG. 3 shows an exemplary and particularly preferred embodiment of a measuring system 30 for a container 1, such as a bioreactor. The measuring system 30 is preferably part of a line system 10 for a container 1 (e.g., corresponding to FIG. 1). The measuring system 30 further comprises a line body 12, which is preferably substantially tubular and/or rectilinear. However, the line body 12 may also have a different design therefrom. In the preferred embodiment shown of the measuring system 30, the measuring system furthermore has a plurality of (e.g., four) measuring apparatuses 38, 40, 42 and 44, each of which is mounted on a coupling apparatus 16 of the line body 12 of the line system 10 or coupled to one of said coupling apparatuses 16 in each case. The measuring apparatuses 38, 40, 42 and 44 are preferably detachably coupled to the coupling apparatuses 16. More preferably, the line body 12 has connection regions 14 at both ends in order to insert or integrate the line body 12 into the line system 10 of a container. Sterile connectors (e.g., Lynx®, Opat®) are preferably used for this purpose in order to achieve a substantially leak-tight connection between the line body 12 and, for example, components of the line system 10 or a container 1. Alternatively and/or additionally, a connector clamp (Tri-Clamp), an adapter, a coupling, a hose barb, a quick connector, a plug-in nipple and/or an external and/or internal thread can be provided, for example, in order to insert the measuring system 30 or the line body 12 into the line system 10. Seals or sealants are preferably used to achieve a substantially leak-tight connection.

A measuring apparatus 32 of the embodiment of the measuring system 30 shown comprises a pressure sensor 38 which is coupled to a deflectable membrane 26 of the line body 12, as shown in FIG. 2. According to a preferred embodiment, the pressure sensor 38 comprises a piezoelectric sensor. The pressure sensor 38 is mounted (preferably detachably) on the coupling apparatus 16 of the line body with a fastening device 36 in such a way that the piezoelectric sensor is mechanically coupled to the deflectable membrane 26 in order to enable transmission of the pressure from the medium inside the line body 12 via the membrane 26 to the pressure sensor 38. Alternative measuring apparatuses for measuring the pressure of the medium comprise, for example, capacitive, inductive and/or piezoresistive sensors.

In the preferred embodiment in FIG. 3, a measuring apparatus 32 comprises a flow sensor 40 for measuring the flow rate of the medium through the line body 12. The exemplary embodiment shown of the flow sensor 40 comprises a housing which completely encloses the line body 12. Housings of the measuring apparatus 32 may also only partially enclose the line body 12. The flow sensor 40 is preferably designed to be mounted on or coupled to a coupling apparatus 16 of the line body, for example by means of a “clamp-on” mechanism. In the region of the coupling apparatus 16 for a flow sensor 40, at least one region of the wall of the line body 12 preferably has a reduced thickness, preferably less than approximately 2 mm, so that ultrasound used for the measurement can arrive at the medium in the line body 12 substantially unimpeded. The housing of the flow sensor 40 may (as shown in FIG. 3) also include other sensors suitable for measuring parameters of the medium flowing or streaming through the line body 12 and/or may also have different designs, for example have a round shape.

A further measuring apparatus 32 of the measuring system 30 shown preferably comprises a conductivity sensor 42, which is preferably coupled to a coupling apparatus 16 of the line body with (preferably four) electrodes 28 integrated into the line body 12 and thus allows a measurement of the conductivity of the medium within the line body 12. The conductivity sensor 42 shown preferably comprises a fastening device 36 for detachable coupling to the coupling apparatus 16 of the line body 12. The conductivity sensor 42 has contact points that are in or come into contact with the electrodes 28 of the coupling apparatus 16. An electrical signal can thus be conducted into the interior of the line body 12 or one or more signals from the interior of the line body 12 can be detected. A particularly preferred embodiment of a receiving apparatus 16 and coupled measuring apparatus 42 is shown in FIG. 5.

In the exemplary embodiment shown in FIG. 3, the measuring apparatuses 38 and 42 each have a fastening device 36 for being coupled to the coupling apparatuses 16 of the line body 12, wherein the coupling preferably takes place in a detachable manner. The fastening devices 36 preferably comprise single-part or multi-part clamps which at least partially enclose the line body 12 on a coupling apparatus 16 or in the vicinity thereof. Alternatively and/or additionally, suitable fastening devices 36 for mounting or fastening or coupling the measuring apparatuses 32 may comprise, for example, cable ties and/or a flange. Fastening devices 36 for measuring apparatuses 32 may also additionally and/or alternatively comprise magnetic and/or other adhesives. The fastening devices 36 mentioned only comprise an exemplary selection of suitable fastening devices 36.

Furthermore, the preferred embodiment of the measuring system 30 shown in FIG. 3 has a coupling apparatus 16 comprising a receiving apparatus 20, which at least partially houses or accommodates a measuring apparatus 32. Such a receiving apparatus 20 is preferably formed integrally with the wall of the line body 12. Optionally, the receiving apparatus 12 is mounted in such a way that a measuring apparatus 32 received by it or a sensor 44 included in the measuring apparatus 32 can be coupled to a region described above of a coupling apparatus 16 having advantageous properties. In the preferred embodiment shown, the measuring apparatus 32 comprises a temperature sensor 44 (e.g., a PT-100 or NTC/PTC probe). Due to the positioning or mounting in the receiving apparatus 20, the temperature sensor 44 is particularly advantageously coupled to the coupling apparatus 16 for measuring the temperature of the medium in the line body 12, since, in particular, a detachment and/or an incorrect mounting of the measuring apparatus 32 can be prevented. Such an advantageous coupling also enables, in particular, an improved mechanical or electrical contact between measuring apparatuses 32 and the line body 12 or the coupling apparatuses 16 so that an improved transmission of, for example, heat and/or sound waves and/or electrical signals can be achieved. In addition, the receiving apparatus 20 of the coupling apparatus 16 also further preferably functions as thermal insulation, so that the measurement accuracy of a temperature measurement at the coupling apparatus 16 can advantageously be increased.

In addition to the preferred embodiments of coupling apparatuses 16 and measuring apparatuses 32 described above, said apparatuses may also have other configurations. An alternative preferred coupling apparatus 16 has, for example, one or more openings in the line body 12 for receiving at least a portion of a measuring apparatus 32 or for inserting at least a portion of a measuring apparatus 32 into the interior of the line body 12. The openings may advantageously be closed in a substantially leak-tight manner and/or comprise a seal so that a measuring apparatus 32 positioned in the opening closes the opening in a substantially leak-tight manner. The openings may preferably be closed, for example, with a plug if no measuring apparatus 32 is mounted on or in said openings or coupled thereto during operation of the measuring system 30.

A further alternative embodiment of a coupling apparatus 16 is a coupling apparatus 16 comprising two opposing openings. Such a coupling apparatus 16 is particularly suitable for a measuring apparatus 32 for measuring viscosity, wherein an acoustic wave resonator is mounted on or in the one opening and an acoustic wave sensor is mounted on or in the opposite opening in order to conduct acoustic waves into or through the medium or to detect at least some of said acoustic waves. A coupling apparatus 16, in particular a coupling apparatus for coupling to an acoustic wave resonator and/or acoustic wave sensor, advantageously has a reinforced wall in order to ensure sufficient resistance to the increased mechanical stress. Such a reinforcement may, in particular, comprise an increase in the wall thickness and/or the mounting or embedding of reinforcing structures.

Measuring apparatuses 32 suitable for the measuring system 30 may have different embodiments, in particular with regard to the receiving and/or transmission of signals. The measuring apparatuses 32 may therefore have different means for connecting the measuring apparatuses 32 or the sensors included therein, for example, to a control unit or evaluation unit and for transmitting signals, in particular measurement signals. Both mechanical contacts and/or plug connections may be provided and wireless transmission of the signals may take place. An optionally required or advantageous power supply may likewise be provided by means of a battery and/or cable connection.

FIG. 4 shows an exemplary and particularly preferred embodiment of a coupling apparatus 16 for a measuring apparatus 32 comprising a temperature sensor 44 (e.g., PT100 or NTC/PTC). FIG. 4 shows a partial segment of a line body 12. The coupling apparatus 16 shown has a receiving apparatus 20, which is preferably designed integrally with the wall of the line body 12. Alternatively and/or additionally, such a receiving apparatus 20 can be a component that is separate from the line body 12 and arranged thereon (preferably separably). In the preferred embodiment shown, the receiving apparatus 20 is arranged in such a way that a measuring apparatus 32 received thereby or the temperature sensor 44 can be coupled to a region having advantageous properties described above. As shown, the receiving apparatus 20 has a guide or a shaft for the measuring apparatus 32 or the temperature sensor 44 in order to couple the temperature sensor 44 to a specific region of the coupling apparatus 16. More preferably, a receiving apparatus 20 has thermal insulation in order to reduce external influences on a received temperature sensor 44.

The particularly preferred embodiment of the coupling apparatus 16 has a region suitable for temperature measurement. Such a region preferably has a thermal conductivity of greater than approximately 100 W/mK, more preferably greater than approximately 200 W/mK, more preferably greater than approximately 300 W/mK, and/or constitutes a heat-conducting bridge. Such a heat-conducting bridge can, for example, comprise a small plate which is inserted or embedded in a substantially leak-tight manner into a cutout in the line body 12 so that the small plate 22 can come into contact with the medium on one side and is simultaneously substantially accessible from outside the line body 12. Such a heat-conducting bridge preferably comprises a material having a high thermal conductivity, such as aluminum, gold, copper, and/or silver. More preferably, such a heat-conducting bridge has a wall thickness of less than approximately 3 mm, more preferably less than approximately 2 mm. The heat-conducting bridge inserted or embedded into the wall of the line body 12 can be sealed, for example, by suitable plastomers and/or elastomers. Alternatively and/or additionally, the heat-conducting bridge can have a structure, for example a lattice structure, integrated into the wall of the line body 12. A lattice structure can be embedded in the wall of the line body 12 or provided therein or thereon, for example, during the manufacturing process of the line body 12, for example an injection molding process, and optionally exposed by cutting/machining so that the lattice structure can come into contact with the medium and/or a measuring apparatus, in particular a temperature sensor 44.

In the particularly preferred embodiment shown, the coupling apparatus 16 comprises a conductive element (preferably small metal plate) 22 embedded in the wall of the line body 12, which conductive element preferably terminates substantially flush with the inner surface of the line body 12 and/or can come into contact with a medium located in the line body 12. The small metal plate 22 preferably has a thickness of less than approximately 2 mm. Furthermore, the coupling apparatus 16 comprises a receiving apparatus 20 for receiving the measuring apparatus 32. In this case, the measuring apparatus 32 is preferably coupled to the coupling apparatus 16 in such a way that the temperature sensor 44 is in contact with the small metal plate 22 in order to enable an advantageous temperature transfer from the medium via the small metal plate 22 to the temperature sensor 44. Alternatively and/or in addition to the depicted small metal plate 22, other elements having different shapes and consisting of different materials can provide advantageous thermal coupling between the temperature sensor 44 and the medium streaming or flowing in the line body 12. In particular, materials which have an increased thermal conductivity relative to the line body 12 are suitable for this purpose. Alternatively and/or additionally, the thickness of the wall of the line body 12 can be reduced in order to achieve advantageous thermal coupling between the temperature sensor 44 and the medium.

FIG. 5 shows a partial segment of a coupling apparatus 16 of the line body 12 and a measuring apparatus 32 according to an exemplary preferred embodiment. The coupling apparatus 16 comprises a wall of the line body 2 having a reduced wall thickness of preferably approximately 2 mm. The coupling apparatus 16 also has a plurality of (preferably four) electrodes 28 which are embedded into the wall of the line body 12 and can be contacted from inside and outside the line body 12. The measuring apparatus 32 coupled to the coupling apparatus 16 comprises a conductivity sensor 42 or a means associated with such for contacting the electrodes 28, for example spring-loaded contacts 43. Alternatively and/or additionally, the electrodes 28 may have structures projecting radially outward, which can preferably be coupled to a measuring apparatus 32 having suitable receiving or contact elements. In the embodiment shown, the measuring apparatus 32 is coupled to the coupling apparatus 16 of the line body 12 by means of a clip 36. The clip 26 preferably comprises structural features which prevent an incorrect mounting of the measuring apparatus to the coupling apparatus 16 or the line body 12, in particular a mounting in which the electrodes 18 are not in contact with means for contacting the measuring apparatus 32. The clip 36 and an associated coupling apparatus 16 are particularly preferably designed in such a way that correct positioning and/or mounting of the clip 36 or of the measuring apparatus 32 on the coupling apparatus 16 can be verified by simple visual inspection.

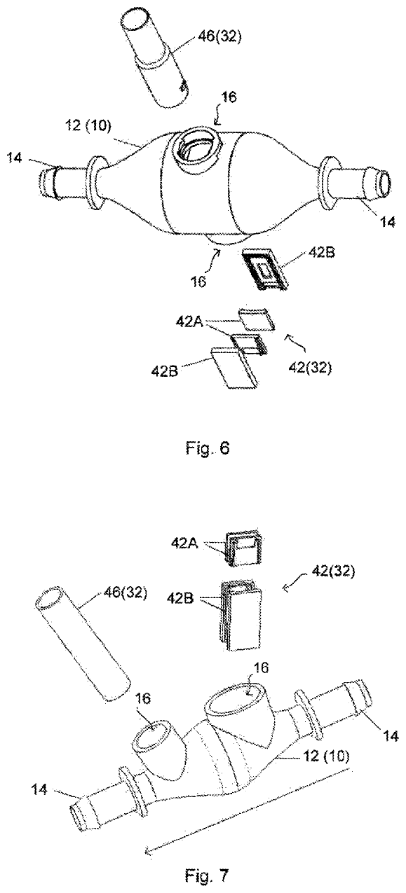

FIG. 6 shows a further exemplary, particularly preferred embodiment of a measuring system 10 of the present invention having two couplable measuring apparatuses 32. In this embodiment, the line body 12 has a plurality of (e.g., two) coupling apparatuses 16, each of which for its part has an opening for receiving at least a portion of a measuring apparatus 32 or for inserting at least a portion of a measuring apparatus 32 into the interior of the line body 12. The openings of the coupling apparatuses 16 can advantageously be closed in a substantially leak-tight manner and/or have a seal so that a measuring apparatus 32 positioned in the opening closes the opening in a substantially leak-tight manner. The openings can preferably be closed, for example, with a plug or a cover, if no measuring apparatus 32 is mounted on or in the openings of the coupling apparatuses 16 or coupled thereto during operation of the measuring system 30.

In the particularly preferred embodiment shown, two coupling apparatuses 16 are arranged opposite each other, i.e., on substantially radially opposite regions of the line body 12. Alternatively, two or more coupling apparatuses 16 can be arranged uniformly or as desired along the circumference of the line body 12. The positions of the coupling apparatuses 16 or parts thereof can be arranged at substantially identically spaced positions relative to the connection regions 14. Alternatively, the positions along the length of the line body 12 can vary to a greater or lesser degree. In particular, the coupling apparatuses 16 can be arranged at any desired offset in order, for example, to satisfy various requirements, such as measuring positions and/or accessibility.

The specific, advantageous arrangement of the coupling apparatuses 16 and the measuring apparatuses 32 enables, in particular, a mutual influence of the different sensors to be prevented or at least reduced. Furthermore, the depicted embodiment advantageously does not have a predetermined alignment of the line body 12 in the line system 10 with respect to the direction of flow of the medium. This ensures simplified handling and assembly of the measuring system 30.

The measuring system 30 shown in FIG. 6 preferably has two measuring apparatuses 32 which are suitable for being coupled to the openings of the coupling apparatus 16 or for being at least partially received by the coupling apparatuses 16. The first measuring apparatus 32 comprises, for example, a pH sensor 46. The second measuring apparatus 32 comprises, for example, a conductivity sensor 42 (shown in exploded view). The coupling apparatuses 16 or their openings and the housings of the measuring apparatuses 32 are advantageously designed in such a way that correct and stable positioning or fixing of the measuring apparatuses 32 or sensors on or in the line body 12 is ensured. This promotes both the obvious compatibility or non-compatibility of measuring apparatuses 32 and coupling apparatuses 12, a substantially leak-tight mounting or coupling of the measuring apparatuses 32, and a prevention, or at least a reduction, of interfering influences on the measurement results.

Furthermore, a reproducible flow pattern of the medium in the respective measuring region of the sensors is advantageous for this and other embodiments of the invention. This may be ensured or facilitated, inter alia, by a corresponding design of the flow channel within the line body 12. An exemplary and particularly preferred design of the flow channel in a measuring region is shown in FIG. 9. An identical, similar or substantially equivalent design can in principle be integrated into each of the embodiments of the invention shown and not shown.

A coupling apparatus 16 of the line body 12 in FIG. 6 is designed to receive a conductivity sensor 42. The exemplary conductivity sensor 42 shown comprises one or more (e.g., two) front plates and rear plates, wherein in each case one front plate 42A and one rear plate 42B form a pair of plates. The pairs of plates of the conductivity sensor 42 shown can be introduced through the opening of the coupling apparatus 42 into the interior of the line body 12 and positioned in the medium flow region 48 in order to measure the conductivity of the medium flowing past. Alternatively, conductivity sensors 42 of different design and/or sensors for measuring further parameters can be mounted, in particular one or more of the aforementioned pressure sensors 38, flow sensors 40 and/or temperature sensors 44. One or more of the coupling apparatuses 16 may also have a coating 18, a receiving apparatus 20, a small metal plate 22, a window 28, a membrane 26 and/or an electrode 28.

FIG. 7 shows a further alternative embodiment of a line body 12. Unlike the line body 12 in FIG. 6, the present line body 12 has a plurality of (e.g., two) coupling apparatuses 16 arranged at a distance from one another in the direction of flow of the medium. In this embodiment, there can preferably be a predetermined assembly orientation so that a specific connection region 14 is defined as the inlet and the other connection region 14 is defined as the outlet. This is particularly advantageous if a pH sensor 46 and a conductivity sensor 42 are respectively coupled to the coupling apparatuses 16. In this embodiment, the conductivity sensor 42 is advantageously arranged closer to the inlet connection region 14 than the pH sensor 46. The pH sensor 46 is preferably arranged on the line body 12 at a distance in the flow direction of the medium. In this way, the individual measurements of the sensors can be carried out without interference, repeatably, continuously and precisely. In particular, this avoids possible influencing of the conductivity measurement, in particular due to ions escaping from a reference electrode of the pH combined electrode or of the pH sensor 46.

In particular, in the case of an assembly specification that is dependent on the direction of flow, the connection regions 14 can be of different designs so that incorrect installation in the line system is prevented, or at least made more difficult.

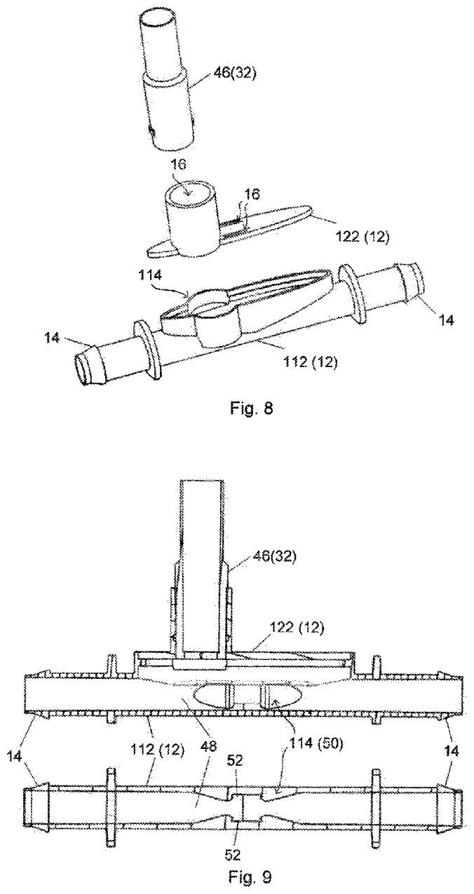

FIG. 8 shows a further preferred embodiment of the invention in which the line body 12 has a line main body 112 and a line sub-body 122. The line main body 112 has a cutout 114 designed in such a way that the line body 122 can be arranged at least partially at or in the cutout 114. The line sub-body 122 comprises one or more (e.g., two) coupling apparatuses 16. The line main body 112 may also have one or more further coupling apparatuses 16. In this advantageous embodiment, it is possible to replace the line sub-body 122 with another line body 122. A line body 12 integrated into a line system 10 can thus be made compatible with other measuring apparatuses 32 or sensors in a simple manner. For example, a line sub-body 122 having coupling apparatuses 16 for a specific pH sensor 46 and a specific conductivity sensor 42 can be replaced by another line sub-body 122 having other coupling apparatuses 16, for example for a pressure sensor 38 and/or a flow sensor 40, without having to remove the line body 12 from the line system 10. The same applies to measuring apparatuses 32, which measure the same parameters but have a different design.

A line sub-body 122 without coupling apparatuses 16 may also serve as a placeholder, wherein the line sub-body 122 without coupling apparatuses 16 is exchanged for a line sub-body 122 having one or more coupling apparatuses 16 as needed in order to be able to carry out measurements. A line body 12 according to this exemplary embodiment has increased versatility since respectively required coupling apparatuses 16 can be replaced and/or retrofitted or supplemented. The arrangement of the individual coupling apparatuses 16 can also be arranged differently, for example as shown in FIG. 6, i.e. at approximately the same distance from a connection region 14.

Advantageously, the cutout 114 and the line sub-body 122 are designed in such a way that a substantially leak-tight mounting of the line sub-body 122 in or on the cutout 144 is made possible at least in regions. An arrangement which does not negatively influence the flow in the interior of the line body 12 is likewise preferred. Moreover, a non-symmetrical design of the cutout 114 and of the line sub-body 122 can be advantageous, since incorrect assembly with respect to the flow direction of the medium can thus be prevented.

FIG. 9 shows two sectional views of an exemplary embodiment of the invention in which the line body 12 comprises a line main body 112 and a line sub-body 122 as described for FIG. 8. Moreover, the line body 12, preferably the line main body 112, comprises a flow-shaping geometry 50 in at least one measuring region of the medium flow. The geometry 50 has a structural design which imparts certain features to the medium flow flowing through the measuring region. A hydrodynamic and/or aerodynamic structure which prevents turbulence and/or backflow and/or dead spaces, or at least significantly reduces them, is preferred. FIG. 9 shows an exemplary, especially preferred embodiment of a flow-shaping geometry 50. Further desired or advantageous flow properties are, for example, flow velocity and flow direction (e.g. deviating from the main flow direction in the line body), which are achieved by corresponding designs.

In the exemplary embodiment in FIG. 9, the flow-shaping geometry 50 is arranged in such a way that the advantageous medium flow it causes is generated in a measuring region in which, for example, a conductivity sensor 42 as shown in FIGS. 6 and 7 can be mounted. The hydrodynamic and/or aerodynamic structures of the flow-shaping geometry 50 shown advantageously have a substantially symmetrical shape. The flow-shaping geometry 50 preferably comprises one or more recesses 52, into which one or more portions of a measuring apparatus 32 or of a sensor can preferably be inserted with a substantially precise fit. In this way, any negative effect on the laminar flow in the measuring region is as small as possible. The described structures of the flow-shaping geometry 50 are exemplary of the conductivity sensor 42 shown in FIGS. 6 and 7. The rear plates 42B sit precisely in the recesses 52 so that a substantially smooth flow channel wall is present in the measuring region. Where designs of the sensors or of the measuring apparatuses 32 differ, correspondingly differing structures of the flow-shaping geometry 50 are advantageous.

Designs and/or arrangements of a flow-shaping geometry 50 may be varied depending on the requirements on the measuring system 30. The flow body 12 can also have a plurality of flow-shaping geometries 50, especially also in conjunction with other coupling apparatuses 16, so that other measuring apparatuses 32 or sensors can also be mounted on an advantageous measuring region. In addition, the measuring apparatuses 32 or sensors can have exclusively or additionally hydrodynamic and/or aerodynamic structures in order to influence the medium flow in their respective measuring regions inside the flow body 12. The same applies to the line sub-body 122.

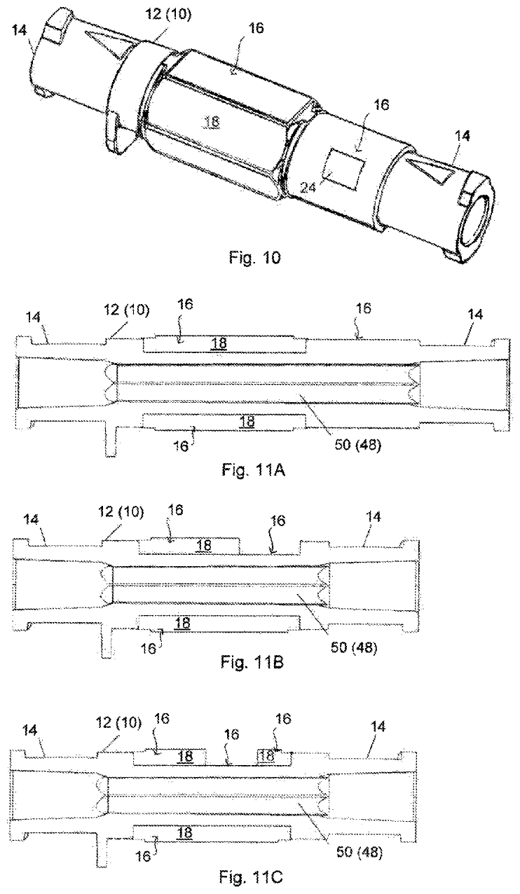

FIG. 10 shows another exemplary embodiment of the invention having two coupling apparatuses 16. The line body 12 and the connection regions 14 preferably have a first material, for example a thermoplastic, such as polybutylene terephthalate (PBT/PTMT), Celanex® or Vestodur®. In the embodiment shown, a first coupling apparatus 16 has a coating 18, which a second material on, which differs from the first material. The second material advantageously comprises a thermoplastic elastomer, e.g., comprising or essentially consisting of TPE (thermoplastic elastomer, such as TPA, TPC, TPO, TPS, TPU, TPV), silicone, NBR (acrylonitrile butadiene rubber) or materials having similar properties. Such a coating or a similar coating enables, in particular, an advantageous mechanical coupling of a measuring apparatus, for example a temperature sensor, to the first coupling apparatus 16.

As shown in FIG. 10, the first coupling apparatus 16 has a polygonal, for example hexagonal, cross section or outer shape. The second material can be provided over the entire outer circumference or over subregions thereof. The outer circumference of the coupling apparatus 16 may also have a substantially round shape.

The second coupling apparatus 16 of the line body 12 shown in FIG. 10 comprises, for example, a substantially round cross-section and an optical window 24, e.g., in the form of a glass substrate or a glass pane or a body which is substantially transparent in the optical range of the electromagnetic spectrum and is made of, for example, sapphire, quartz, MgF2, CaF2, Ge, Si, diamond. Instead of or in addition to the embodiment of the second coupling apparatus 16 shown, other embodiments of the coupling apparatuses 16 disclosed in this application can also be provided, in particular a coupling apparatus 16 having small metal plates 22, membrane 26 or electrode(s) 28.

FIG. 11A shows a longitudinal section of a line body 12 according to FIG. 10. In the particularly preferred embodiment shown, the coating 18 of the first coupling apparatus 16 is embedded in a recess in the line body 12. Parameters of the coating 18 may vary as required—in particular, thickness, number of layers, layer material(s), length, width, and/or surface condition. The line body 12 shown also has a flow-shaping geometry 50 in the medium flow region 48 of the one or more coupling apparatuses 16 (as described for FIG. 9). As shown in FIG. 11A, the line body 12 may optionally have an additional, third coupling apparatus 16, for example on the opposite side of the first coupling apparatus 16. With the first and third coupling apparatuses 16 shown, for example, a particularly advantageous redundancy measurement having two measuring apparatuses 32 of the same type can take place at the same location in the medium flow area 48.

FIG. 11B shows a longitudinal section of an alternative embodiment of the line body 12 shown in FIGS. 10 and 11A. The present exemplary embodiment differs in particular in the second coupling apparatus 16, which is arranged in a region of the line body 12 having reduced diameter. In this example, the second coupling apparatus 16 is located in a recess in the coating 18 of the first coupling apparatus 16 as shown in FIGS. 10 and 11A and is particularly suitable for coupling a temperature sensor. The recess shown in the coating 18 can be effected, for example, by omitting this region when applying a coating 18 or by (at least partially) removing a previously applied coating 18. Such an embodiment is characterized in particular by a compact design and simplified production. In addition, an interfering influence of a temperature measurement can be prevented, at least considerably reduced, by corresponding properties of the second material (for example, temperature-insulating/weakly temperature-conducting). The second coupling apparatus 16 may moreover have a coating with a third material and/or a different form to the first coupling apparatus 16.

FIG. 11C shows a further alternative embodiment in which the second coupling apparatus 16 is provided between the first and an optional additional fourth coupling apparatus 16, wherein the fourth coupling apparatus 16 is preferably identical to the first coupling apparatus 16. In this way, a further measuring apparatus 32 can be coupled to the line body 12, while maintaining the compact size of the line body 12.

The line bodies 12 in FIGS. 11B and 110 likewise preferably have a flow-shaping geometry 50 in the medium flow region 48. In addition, the line body 12 in FIGS. 10-11C may alternatively or additionally have further identical and/or differently designed coupling apparatuses 16 as well as further features of the embodiments described in this application.

### LIST OF REFERENCE SIGNS

1 Container (e.g., bioreactor)
4 Medium
6 Valve
8 Stirring device
10 Line system
12 Line body
14 Connection region
16 Coupling apparatus
18 Coating
20 Receiving apparatus
22 Small metal plates
24 Window
26 Membrane
28 Electrode
30 Measuring system
32 Measuring apparatus
36 Fastening device
38 Pressure sensor
40 Flow sensor
42 Conductivity sensor
42A Front plate
42B Rear plate
43 Spring-loaded contact
44 Temperature sensor
46 pH sensor
48 Medium flow region
50 Flow-shaping geometry
52 Recess
112 Line main body
114 Cutout
122 Line sub-body

1.-19. (canceled)

20. A container for storing, mixing and/or cultivating a medium, the container comprising a line system as a discharge line, feed line and/or bypass line of the container, the line system at least comprising:
a line body formed as a single piece and configured for a medium to flow therethrough, the line body having:
a first and a second connection region for connecting in particular to the container and/or the line system; and
at least a first and a second coupling apparatus in the region between the connection regions, the first and second coupling apparatuses being structurally different and being configured to be coupled to respective structurally different measuring apparatuses.

21. The container according to claim 20, wherein the line body has at least a third coupling apparatus, and, preferably, wherein the first, second and third coupling apparatuses are structurally different.

22. The container according to claim 20, wherein the coupling apparatuses are arranged at a distance from one another in a circumferential direction of the line body and/or in the flow direction of a medium flowing through the line body.

23. The container according to claim 20, wherein the measuring apparatuses are configured to measure one or more of a temperature, pressure, flow rate, oxygen content, pH value, conductivity, viscosity and/or optical parameters of a medium flowing through the line body.

24. The container according to claim 20, wherein the line body is a single-use line body.

25. A line body for a line system for discharging, feeding and/or diverting a medium of a container for storing, mixing and/or cultivating a medium, wherein the line body is formed as a single piece and has:
a first and a second connection region for connecting the line body in particular to a container and/or to the line system; and
at least a first and a second coupling apparatus in the region between the connection regions (14), the first and second coupling apparatuses being structurally different and being designed to be coupled to respective structurally different measuring apparatuses.

26. The line body according to claim 25, wherein the line body has at least a third coupling apparatus, wherein the first, second and third coupling apparatuses of the line body are structurally different.

27. The line body according to claim 25, wherein the measuring apparatuses are configured to measure one or more of a temperature, pressure, flow rate, oxygen content, pH value, conductivity, viscosity and/or optical parameters of a medium flowing through the line body.

28. The line body according to claim 25, wherein the line body is a single-use line body.

29. The line body according to claim 25, wherein at least one region of a coupling apparatus of the line body has:
a wall thickness of less than approximately 3 mm, preferably less than approximately 2 mm; and/or
a coating on the outside of the line body; and/or
a thermal conductivity to enable measurements of a temperature of the medium from outside the line body; and/or
an optical transmittance to enable measurements of optical parameters of the medium from outside the line body; and/or
sound permeability to enable measurements of a flow rate of the medium from outside the line body.

30. The line body according to claim 25, wherein a coupling apparatus of the line body has one or more openings in the line body for receiving at least a portion of a measuring apparatus or for inserting at least a portion of a measuring apparatus into an interior of the line body.

31. The line body according to claim 25, wherein a coupling apparatus of the line body has a deflectable membrane in order to enable measurements of a pressure within the line body or the medium from outside the line body, wherein the deflectable membrane has:
a first side which faces an interior of the line body and can come into contact with the medium inside the line body; and
a second side accessible from outside the line body.

32. The line body according to claim 25, wherein a coupling apparatus of the line body comprises two or more electrodes, and wherein:
a first side of the two or more electrodes faces an interior of the line body and can come into contact with the medium inside the line body; and
a second side of the two or more electrodes is accessible from outside the line body.

33. The line body according to claim 25, wherein a coupling apparatus has a receiving apparatus configured to receive at least a portion of a measuring apparatus.

34. The line body according to claim 33, wherein the receiving apparatus is a shaft and/or a rail.

35. The line body according to claim 25, wherein the line body is suitable for radiation sterilization and/or autoclave sterilization or steam sterilization.

36. The line body according to claim 25, wherein coupling apparatuses are arranged at a distance from one another in the direction of flow of a medium flowing through the line body and/or in a circumferential direction of the line body.

37. The line body according to claim 25, further comprising a flow-shaping geometry in a medium flow region of the line body for influencing the flow properties of the medium.

38. The line body according to claim 25, wherein the line body comprises:
a line main body having the connection regions and a cutout, and
a line sub-body having at least two coupling apparatuses,
wherein the cutout of the line main body is designed to receive the line sub-body at least in regions.

39. A measuring system for a container for storing, mixing and/or cultivating a medium, in particular a bioreactor, for measuring parameters of a medium, comprising:
a line body according to claim 25; and
two or more measuring apparatuses, each of which is coupled to one of the coupling apparatuses of the line body, and wherein at least two of the measuring apparatuses are structurally different.
